(19) United States
(12) Patent Application Publication
(10) Pub. No.: US 2022/0023400 A1
(43) Pub. Date: Jan. 27, 2022

(54) ARTIFICIAL PROMISCUOUS T HELPER CELL EPITOPES THAT FACILITATE TARGETED ANTIBODY PRODUCTION WITH LIMITED T CELL INFLAMMATORY RESPONSE

(71) Applicant: UBI US Holdings, LLC., Hauppauge, NY (US)

(72) Inventor: Chang Yi WANG, Cold Spring Harbor, NY (US)

(21) Appl. No.: 17/053,059

(22) PCT Filed: May 3, 2019
(86) PCT No.: PCT/US19/30649

§ 371 Date: Nov. 4, 2020

Related U.S. Application Data
(60) Provisional application No. 62/667,123, filed on May 4, 2018.

(51) Int. Cl. A61K 39/00; A61P 31/14

(52) U.S. Cl. CPC A61K 39/0007; A61K 39/39; A61P 31/14

(57) ABSTRACT

The present invention is directed to novel heterologous promiscuous and artificial T helper cell epitopes (Th epitopes) designed to provide optimum immunogenicity of a target antigenic site. The disclosed Th epitopes, when covalently linked to a B cell epitope in a peptide immunogen construct, elicit a strong antibody response to the B cell epitope of the target antigenic site. The Th epitopes are immunosilent on their own, i.e., little, if any, of the antibodies generated by the peptide immunogen constructs will be directed towards the Th epitope, thus allowing a very focused immune response directed to the targeted antigenic site. The heterologous promiscuous Th epitopes provide effective and safe peptide immunogens that do not generate inflammatory, anti-self, cell-mediated immune responses following administration.

The present application is a PCT International Application that claims the benefit of U.S. Provisional Application Ser. No. 62/667,123, filed May 4, 2018, which is incorporated herein by reference in its entirety.

### BACKGROUND

Immune responses require the cooperative interaction between antigen-presenting cells and T helper cells. The elicitation of an effective antibody response requires that antigen-presenting cells recognize the target antigenic site of a subject immunogen and that the T helper cells recognize a T helper cell epitope. Generally, the T helper epitope on a subject immunogen is different from its B cell epitope(s). The B cell epitope is a site on a desired target that is recognized by B cells, which results in the production of antibodies against the desired target site. The natural conformation of the target determines the site to which the antibody directly binds. Evocation of a Th cell response requires a Th cell receptor to recognize a complex on the membrane of an antigen-presenting cell that is formed between a processed peptide fragment of a target protein and an associated class II major histocompatibility complex (MHC). Thus, peptide processing of the target protein and three-way recognition are required for a Th cell response. The three part complex is difficult to define because 1) the critical MHC class II contact residues are variably positioned within different MHC binding peptides (Th epitopes); 2) the different MHC binding peptides have variable lengths and different amino acid sequences; and 3) MHC class II molecules can be highly diverse depending on the genetic make-up of the host. The immune responsiveness to a particular Th epitope is in part determined by the MHC genes of the host, and the reactivity of Th epitopes differs among individuals of a population. Promiscuous Th epitopes, i.e., Th epitopes that are reactive across species and individuals within a single species, are difficult to identify.

Multiple factors are required for each component step of T cell recognition, such as appropriate peptide processing by the antigen-processing cell, presentation of the peptide by a genetically determined class II MHC molecule, and recognition of an MHC molecule or peptide complex by the receptor on Th cells. The requirements for promiscuous Th epitope recognition for providing broad responsiveness can be difficult to determine.

It is clear that for the induction of antibodies, the immunogen must comprise both the B cell determinant and Th cell determinant(s). Commonly, to increase the immunogenicity of a target, the Th response is provided by coupling the target to a carrier protein. The disadvantages of this technique are many. It is difficult to manufacture well-defined, safe, and effective peptide-carrier protein conjugates for the following reasons:

a. Chemical coupling are random reactions introducing heterogeneity of size and composition, e.g., conjugation with glutataraldehyde (Borras-Cuesta et al., Eur J Immunol, 1987; 17: 1213-1215);

b. the carrier protein introduces a potential for undesirable immune responses such as allergic and autoimmune reactions (Bixler et al., WO 89/06974);

c. the large peptide-carrier protein elicits irrelevant immune responses predominantly misdirected to the carrier protein rather than the target site (Cease et al., Proc Natl Acad Sci USA, 1987; 84: 4249-4253); and

d. the carrier protein also introduces a potential for epitopic suppression in a host which had previously been immunized with an immunogen comprising the same carrier protein. When a host is subsequently immunized with another immunogen wherein the same carrier protein is coupled to a different hapten, the resultant immune response is enhanced for the carrier protein but inhibited for the hapten (Schutze et al., J Immunol, 1985; 135: 2319-2322).

To avoid the risks described above, it is desirable to elicit T cell help without the use of traditional carrier proteins.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWING

FIG. 1: Detection of promiscuous and artificial Th peptide responsive T cells in naïve Peripheral blood mononuclear cells of normal donors.

### SUMMARY OF THE INVENTION

The present disclosure provides promiscuous artificial T helper cell (Th) epitopes that can be used to produce peptide immunogens that are capable of stimulating functional site-directed antibody responses for therapeutic effects. The disclosed artificial Th epitopes can be linked to a synthetic peptide B cell epitope (“target antigenic site”), through an optional spacer, to produce an immunogenic peptide. The immunogenic peptides can also comprise other components, including a general immune stimulator sequence.

The artificial Th epitope imparts to the peptide immunogen the capability to induce a strong T helper cell-mediated immune response with the production of a high level of antibodies directed against the “target antigenic site.” The present invention further provides for the advantageous replacement of carrier proteins and pathogen-derived T helper cell sites in established peptide immunogens with artificial Th epitopes designed specifically to improve their immunogenicity. The short peptide immunogens with the artificial Th epitopes of the present invention elicit a high level of antibodies targeted to specific target antigenic site B cell epitopes without causing a significant inflammatory response.

The artificial Th epitopes of the present invention can be linked to target antigenic sites and optionally to an immunostimulatory sequence. The immunogenic peptides of the present invention may be represented by the formulae:

(A)ₙ-(Target antigenic site)-(B)ₒ-(Th)ₘ-X

or

(A)ₙ-(B)ₒ-(Th)ₘ-(B)ₒ-(Target antigenic site)-X

or

(A)ₙ-(Th)ₘ-(B)ₒ-(Target antigenic site)-X

or

(Target antigenic site)-(B)ₒ-(Th)ₘ-(A)ₙ-X

or

(Th)ₘ-(B)ₒ-(Target antigenic site)-(A)ₙ-X

wherein:
each A is independently an amino acid;
each B is independently an amino acid, —NHCH(X)CH₂SCH₂CO—, —NHCH(X)CH₂SCH₂CO(εN)Lys-, —NHCH(X)CH₂S-succinimidyl(εN)Lys-, or —NHCH(X)CH₂S-(succinimidyl)-;
each Th is independently an artificial Th cell epitope, an analog, or segment thereof; Target antigenic site is a B cell epitope, a peptide hapten, or an immunologically reactive analogue thereof;
X is an amino acid, α-COOH, or —CONH₂;
n is 1, 2, 3, 4, 5, 6, 7, 8, 9, or 10;
m is 1, 2, 3, or 4; and
o is 0, 1, 2, 3, 4, 5, 6, 7, 8, 9, or 10.

An example of a peptide hapten as a target antigenic site is amino acids 1-14 of the beta-amyloid (Aβ) protein (Aβ₁₋₁₄) (SEQ ID NO: 56).

The compositions of the present invention comprise peptides capable of evoking antibody responses in an immunized host to a desired target antigenic site. The target antigenic site may be derived from pathogenic organisms and normally immunosilent self-antigens and tumor-associated targets.

Accordingly, the compositions of the present invention are useful in many diverse medical and veterinary applications. These include vaccines to provide protective immunity from infectious disease, immunotherapies for the treatment of disorders resulting from the malfunction of normal physiological processes, immunotherapies for the treatment of cancer, and agents to desirably intervene in and modify normal physiological processes.

Some of the targets antigens that may be covalently linked to the Th epitopes of the present invention include portions of: beta-amyloid (Aβ) for the treatment of Alzheimer's Disease, alpha-synuclein (α-Syn) for the treatment of Parkinson's Disease, the extracellular membrane-proximal domain of membrane-bound IgE (or IgE EMPD) for the treatment of allergic disease, Tau for the treatment of tauopathies including Alzheimer's Disease, and Interleukin-31 (IL-31) for the treatment of atopic dermatitis, to name a few. More specifically, Aβ₁₋₁₄ (as described in U.S. Pat. No. 9,102,752), α-Syn126-135 (as described in U.S. Provisional Application No. 62/521,287), IgE EMPD₁₋₃₉ (as described in International PCT Application No. PCT/US2017/069174), Tau₃₇₉₋₄₀₈ (as described in U.S. Provisional Application No. 62/578,124), and IL-31₉₇₋₁₄₄ (as described in U.S. Provisional Application No. 62/597,130).

In some embodiments, the invention provides a method of treating a condition comprising administering a peptide to a subject in need thereof, wherein the peptide comprises a T helper cell epitope and an antigen-presenting epitope, wherein the peptide produces an immunogenic inflammatory response that is at least about 3-fold lower than an immunogenic inflammatory response of a positive control.

### DETAILED DESCRIPTION

The present disclosure provides promiscuous artificial T helper cell (Th) epitopes that can be used to produce peptide immunogens that are capable of stimulating functional site-directed antibody responses for therapeutic effects. The disclosed artificial Th epitopes can be linked to a synthetic peptide B cell epitope (“target antigenic site”), through an optional spacer, to produce an immunogenic peptide.

The artificial Th epitope imparts to the peptide immunogen the capability to induce a strong T helper cell-mediated immune response with the production of a high level of antibodies directed against the “target antigenic site.” The present invention further provides for the advantageous replacement of carrier proteins and pathogen-derived T helper cell sites in established peptide immunogens with artificial Th epitopes designed specifically to improve their immunogenicity. The short peptide immunogens with the artificial Th epitopes of the present invention elicit a high level of antibodies targeted to specific target antigenic site B cell epitopes without causing a significant inflammatory response.

The peptide immunogens of the disclosure can evoke antibody responses in an immunized host against a desired target antigenic site. In some embodiments, the antigenic site is taken from a pathogenic organism (e.g., FMDV VP1, PRRSV GP5, etc.). In some embodiments, the antigenic site is taken from normally immunosilent self-antigens or tumor-associated targets (e.g., Aβ, Tau, Alpha Synuclein, IgE EMPD, IL-31, etc.).

The disclosure describes artificial Th epitopes that can be used to provide peptide immunogens that elicit antibodies targeted to a specific protein. The target antigenic site can include any amino acid sequence from any target peptide or protein. In some embodiments, the disclosure describes artificial Th epitopes that can be used to provide peptide immunogens that elicit antibodies targeted to amyloid β (Aβ), foot-and-mouth disease (FMD) capsid protein, a glycoprotein from porcine reproductive and respiratory syndrome virus (PRRSV), Luteinizing Hormone-Releasing Hormone (LHRH), and any other peptide or protein sequence.

The peptides of the invention can be useful in medical and veterinary applications. In some embodiments, the peptides of the invention can be used as vaccines to provide protective immunity from infectious diseases or neurodegenerative diseases, treat disorders resulting from malfunctioning normal physiological processes, as immunotherapies for treating cancer, and as agents to intervene in normal physiological processes.

### Peptide Immunogens

The term “peptide immunogen” as used herein refers to molecules comprising Th epitopes covalently linked to a target antigenic site through conventional peptide bonds so as to form a single larger peptide or through other forms of covalent linkages, such as a thioester.

The disclosure provides peptide immunogens and compositions comprising peptide immunogens. In some embodiments, an immunogenic peptide comprises an artificial heterologous Th epitope, a target antigenic site containing a B cell epitope, and an optional heterologous spacer.

The presence of an artificial Th epitope in a peptide immunogen can induce a strong Th cell-mediated immune response. In some embodiments, the presence of an artificial heterologous Th epitope in an immunogenic peptide can produce a high level of antibodies directed to a target antigenic site. In some embodiments, the disclosure describes the advantageous replacement of carrier proteins and pathogen-derived Th cell sites in established peptide immunogens with artificial heterologous Th cell epitopes designed to improve immunogenicity. In some embodiments, a peptide immunogen with an artificial Th epitope can elicit a high level of antibody production targeted to the B cell epitope (e.g., Aβ, Tau, Alpha Synuclein, IgE EMPD, IL-31, etc.).

In some embodiments, the immunogenic peptides of the disclosure can be represented by the formulae:

(A)ₙ-(Target antigenic site)-(B)ₒ-(Th)ₘ-X

or

(A)ₙ-(B)ₒ-(Th)ₘ-(B)ₒ-(Target antigenic site)-X

or

(A)ₙ-(Th)ₘ-(B)ₒ-(Target antigenic site)-X

or

(Target antigenic site)-(B)ₒ-(Th)ₘ-(A)ₙ-X

or

(Th)ₘ-(B)ₒ-(Target antigenic site)-(A)ₙ-X

wherein:
each A is independently an amino acid;
each B is independently an amino acid, —NHCH(X)CH₂SCH₂CO—, —NHCH(X)CH₂SCH₂CO(εN)Lys-, —NHCH(X)CH₂S-succinimidyl(εN)Lys-, or —NHCH(X)CH₂S-(succinimidyl)-;
each Th is independently an artificial Th cell epitope, an analog, or segment thereof, Target antigenic site is a B cell epitope, a peptide hapten, or an immunologically reactive analogue thereof;
X is an amino acid, α-COOH, or —CONH₂;
n is 1, 2, 3, 4, 5, 6, 7, 8, 9, or 10;
m is 1, 2, 3, or 4; and
o is 0, 1, 2, 3, 4, 5, 6, 7, 8, 9, or 10.

The peptide immunogens of the disclosure can comprise about 25, about 30, about 35, about 40, about 45, about 50, about 55, about 60, about 65, about 70, about 75, about 80, about 85, about 90, about 95, or about 100 amino acid residues. In some embodiments, the peptide immunogens of the disclosure can comprise about 20, about 30, about 40, about 50, about 60, about 70, or about 80 amino acid residues.

### A—Amino Acid

Each A in the immunogenic peptides of the disclosure is independently a heterologous amino acid sequence.

The term “heterologous”, as used herein, refers to an amino acid sequence that is not part of, or homologous with, the wild-type amino acid sequence of the target antigenic site (B cell epitope). Thus, a heterologous amino acid sequence of A contains an amino acid sequence that is not naturally found in the protein or peptide of the target antigenic site. Since the sequence of component A is heterologous to the target antigenic site, the natural amino acid sequence of target antigenic site is not extended in either the N-terminal or C-terminal directions when component A is covalently linked to the target antigenic site.

In some embodiments, each A is independently a non-naturally occurring or naturally occurring amino acid.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

In some embodiments, n is greater than one, and each A is independently the same amino acid. In some embodiments, n is greater than one, and each A is independently a different amino acid.

### B—Optional Heterologous Spacer

Each B in the immunogenic peptide of the disclosure is an optional heterologous spacer.

As discussed above, term “heterologous” refers to an amino acid that is not part of, or homologous with, the wild-type amino acid sequence of the target antigenic site (B cell epitope). Thus, when the spacer is an amino acid, the spacer contains an amino acid sequence that is not naturally found in the protein or peptide of the target antigenic site. Since the sequence of component B is heterologous to the target antigenic site, the natural amino acid sequence of target antigenic site is not extended in either the N-terminal or C-terminal directions when component B is covalently linked to the target antigenic site.

The optional heterologous spacer of component B is independently an amino acid, —NHCH(X)CH₂SCH₂CO—, —NHCH(X)CH₂SCH₂CO(cN)Lys-, —NHCH(X)CH₂S-succinimidyl(εN)Lys-, —NHCH(X)CH₂S-(succinimidyl)-, and/or any combination thereof. The spacer can contain one or more naturally or non-naturally occurring amino acid residues as described above for component A.

The spacer can be a flexible hinge spacer to enhance the separation of a Th epitope and the target antigenic site. In some embodiments, a flexible hinge sequence can be proline rich. In certain embodiments, the flexible hinge has the sequence Pro-Pro-Xaa-Pro-Xaa-Pro (SEQ ID NO: 55), which is modeled from the flexible hinge region found in immunoglobulin heavy chains. Xaa therein can be any amino acid. In some embodiments, Xaa is aspartic acid. In some embodiments, the conformational separation provided by a spacer can permit more efficient interactions between a presented peptide immunogen and appropriate Th cells and B cells. Immune responses to the Th epitope can be enhanced to provide improved immune reactivity.

When o>1, each B is independently the same or different. In some embodiments, B is Gly-Gly, Pro-Pro-Xaa-Pro-Xaa-Pro (SEQ ID NO: 55), εNLys, εNLys-Lys-Lys-Lys (SEQ ID NO: 53), Lys-Lys-Lys, —NHCH(X)CH₂SCH₂CO—, —NHCH(X)CH₂SCH₂CO(εNLys)-, —NHCH(X)CH₂S-succinimidyl-εNLys-, or —NHCH(X)CH₂S-(succinimidyl)-, and/or any combination thereof.

Exemplary heterologous spacers are shown in Table 2.

### Target Antigenic Site

The disclosure describes artificial Th epitopes that can be used to provide peptide immunogens that elicit antibodies targeted to a specific protein. The target antigenic site can include any amino acid sequence from any target peptide or protein, including foreign- or self-peptides or proteins.

In some embodiments, the disclosure describes artificial Th epitopes that can be used to provide peptide immunogens that elicit antibodies targeted to luteinizing hormone-releasing hormone (LHRH) (e.g., U.S. Pat. Nos. 6,025,468, 6,228,987, 6,559,282, and US Publication No. US2017/0216418); amyloid β (Aβ) (e.g., U.S. Pat. Nos. 6,906,169, 7,951,909, 8,232,373, and 9,102,752); foot-and-mouth disease capsid protein (e.g., U.S. Pat. Nos. 6,048,538, 6,107,021, and US Publication No. 2015/0306203); HIV virion epitopes for prevention and treatment of HIV infection (e.g., U.S. Pat. Nos. 5,912,176, 5,961,976, and 6,090,388); a capsid protein from porcine circovirus type 2 (PCV2) (e.g., US Publication No. 2013/0236487), a glycoprotein from porcine reproductive and respiratory syndrome virus (PRRSV) (e.g., US Publication No. 2014/0335118), IgE (e.g., U.S. Pat. Nos. 7,648,701 and 6,811,782), alpha-synuclein (α-Syn) (U.S. Provisional Application No. 62/521,287), the extracellular membrane-proximal domain of membrane-bound IgE (or IgE EMPD) (International PCT Application No. PCT/US2017/069174), Tau (U.S. Provisional Application No. 62/578,124), and Interleukin-31 (IL-31) (U.S. Provisional Application No. 62/597,130), the CS antigen of plasmodium for prevention of malaria; CETP for prevention and treatment of arteriosclerosis; and any other peptide or protein sequence. All of the patents and patent publications are herein incorporated by references in their entireties.

Exemplary target antigenic sites are shown in Table 3.

### Th—T Helper Epitope

The Th epitope in the peptide immunogen construct enhances the immunogenicity of the target antigenic site, which facilitates the production of specific high titer antibodies directed against the optimized target B cell epitope through rational design.

In some embodiments, the Th epitope is a heterologous sequence. As discussed above, the term “heterologous” refers to an amino acid sequence that is derived from an amino acid sequence that is not part of, or homologous with, the wild-type sequence of the target antigenic site. Thus, a heterologous Th epitope is a Th epitope derived from an amino acid sequence that is not naturally found in the target antigenic site. Since the Th epitope is heterologous to the target antigenic site, the natural amino acid sequence of the target antigenic site is not extended in either the N-terminal or C-terminal directions when the heterologous Th epitope is covalently linked to the target antigenic site.

The Th epitope can have an amino acid sequence derived from any species (e.g., human, pig, cattle, dog, rat, mouse, guinea pigs, etc.). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the peptide immunogen constructs will be directed towards the Th epitope, thus allowing a very focused immune response directed to the targeted antigenic site.

Th epitopes can range in size from approximately 15 to approximately 50 amino acid residues. In some embodiments, Th epitopes can have about 15, about 20, about 25, about 30, about 35, about 40, about 45, or about 50 amino acid residues. Th epitopes can share common structural features and specific landmark sequences. In some embodiments, Th epitopes have amphipathic helices, i.e., alpha-helical structures with hydrophobic amino acid residues dominating one face of the helix and charged and polar resides dominating the surrounding faces.

The Th epitopes and disclosures of WO 1999/066957, and corresponding U.S. Pat. No. 6,713,301, are incorporated herein by reference in their entireties.

A promiscuous Th determinant can be effective in potentiating a poorly immunogenic peptide. Well-designed promiscuous Th/B cell epitope chimeric peptides can elicit Th responses with antibody responses targeted to the B cell site in most members of a genetically diverse population. In some embodiments, Th cells can be supplied to a target antigen peptide by covalently binding a peptide-carrier to a well-characterized promiscuous Th determinant.

Promiscuous Th epitopes can contain additional primary amino acid patterns. In some embodiments, promiscuous Th epitopes can contain a Rothbard sequence, wherein the promiscuous Th epitope contains a charged residue (e.g., -Gly-), followed by two to three hydrophobic residues, followed by a charged or polar residue (Rothbard and Taylor, EMBO J, 1988; 7:93-101). Promiscuous Th epitopes can obey the 1, 4, 5, 8 rule, wherein a positively charged residue is followed by hydrophobic residues at the fourth, fifth and eighth positions, consistent with an amphipathic helix having positions 1, 4, 5 and 8 located on the same face. In some embodiments, the 1, 4, 5, 8 pattern of hydrophobic and charged and polar amino acids can be repeated within a single Th epitope. In some embodiments, a promiscuous T cell epitope can contain at least one of a Rothbard sequence or an epitope that obeys the 1, 4, 5, 8 rule. In other embodiments, the Th epitope contains more than one Rothbard sequence.

Promiscuous Th epitopes derived from pathogens include, but are not limited to: a hepatitis B surface Th cell epitope (HBsAg Th), hepatitis B core antigen Th cell epitope (HBc Th), pertussis toxin Th cell epitope (PT Th), tetanus toxin Th cell epitope (TT Th), measles virus F protein Th cell epitope (MVF Th), Chlamydia trachomatis major outer membrane protein Th cell epitope (CT Th), diphtheria toxin Th cell epitope (DT Th), Plasmodium falciparum: circumsporozoite Th cell epitope (PF Th), Schistosoma mansoni triose phosphate isomerase Th cell epitope (SM Th), and a Escherichia coli TraT Th cell epitope (TraT Th), Clostridium tetani, Bordetella pertussis, Cholera Toxin, Influenza MP1, Influenza NSP1, Epstein Barr virus (EBV), Human cytomegalovirus (HCMV). Examples of Th epitopes used in the present disclosure are shown in Table 1.

In some embodiments, the Th epitopes of the disclosure can be combinatorial Th epitopes containing a mixture of peptides containing similar amino acid sequences. Structured synthetic antigen libraries (SSALs), also referred to as combinatorial artificial Th epitopes, comprise a multitude of Th epitopes with amino acid sequences organized around a structural framework of invariant residues with substitutions at specific positions. The sequences of SSAL epitopes are determined by retaining relatively invariant residues and varying other residues to provide recognition of the diverse MHC restriction elements. Sequences of SSAL epitopes can be determined by aligning the primary amino acid sequence of a promiscuous Th, selecting and retaining residues responsible for the unique structure of the Th peptide as the skeletal framework, and varying the remaining residues in accordance with known MHC restriction elements. Invariant and variable positions with preferred amino acids of MHC restriction elements can be used to obtain MHC-binding motifs, which can be used to design a SSAL of Th epitopes.

The heterologous Th epitope peptides presented as a combinatorial sequence, contain a mixture of amino acid residues represented at specific positions within the peptide framework based on the variable residues of homologues for that particular peptide. In some embodiments, the Th epitope library sequences are designed to maintain the structural motifs of a promiscuous Th epitope and to accommodate reactivity to a wider range of haplotypes. In some embodiments, a member of a SSAL can be the degenerate Th epitope SSAL1 Th1, modeled after a promiscuous epitope taken from the F protein of the measles virus (e.g., SEQ ID NOs: 1-5). In other embodiments, a member of a SSAL can be the degenerate Th epitope SSAL2 Th2, modeled after a promiscuous epitope taken from HBsAg1 (e.g., SEQ ID NOs: 19-24).

The total number of peptides present in a mixture of combinatorial artificial Th epitopes (or SSAL) after synthesis can be calculated by multiplying the number of options available at each variable position together. For example, SEQ ID NO: 16 represents a combination of 32 different peptides because it contains 5 variable positions, where each variable position has an option of 2 different residues (i.e., 2×2×2×2×2=2⁵=32). Similarly, SEQ ID NO: 5 represents a combination of 524,288 different peptides (i.e., 2×4×2×4×2×4×4×4×2×4×2×4=2⁵×4⁷=524,288). The combinatorial artificial Th epitope sequences include (a) the mixture of all the peptides encompassed by the variable sequences and (b) each individual peptide containing a single-sequence within the combination.

In some embodiments, a charged residue Glu or Asp can be added at position 1 to increase the charge surrounding the hydrophobic face of the Th. In some embodiments, the hydrophobic face of an amphipathic helix can be maintained by hydrophobic residues at 2, 5, 8, 9, 10, 13 and 16. In some embodiments, amino acid residues at 2, 5, 8, 9, 10, and 13 can be varied to provide a facade with the capability of binding to a wide range of MHC restriction elements. In some embodiments, variation in amino acid residues can enlarge the range of immune responsiveness of the artificial Th epitopes.

Artificial Th epitopes can incorporate all properties and features of known promiscuous Th epitopes. In some embodiments, the artificial Th epitopes are members of an SSAL. In some embodiments, an artificial Th site can be combined with peptide sequences taken from self-antigens and foreign antigens to provide enhanced antibody responses to site-specific targets. In some embodiments, an artificial Th epitope immunogen can provide effective and safe antibody responses, exhibit high immunopotency, and demonstrate broad reactive responsiveness.

Idealized artificial Th epitopes are also provided. These idealized artificial Th epitopes are modeled on two known natural Th epitopes and SSAL peptide prototypes, disclosed in WO 95/11998. The SSALS incorporate combinatorial MHC molecule binding motifs (Meister et al., 1995) intended to elicit broad immune responses among the members of a genetically diverse population. The SSAL peptide prototypes were designed based on the Th epitopes of the measles virus and hepatitis B virus antigens, modified by introducing multiple MHC-binding motifs. The design of the other Th epitopes were modeled after other known Th epitopes by simplifying, adding, and/or modifying, multiple MHC-binding motifs to produce a series of novel artificial Th epitopes. The promiscuous artificial Th sites were incorporated into synthetic peptide immunogens bearing a variety of target antigenic sites. The resulting chimeric peptides were able to stimulate effective antibody responses to the target antigenic sites.

The prototype artificial helper T cell (Th) epitope shown in Table 1 as “SSAL1 TH1”, a mixture of four peptides (SEQ ID NOs: 1-4) is an idealized Th epitope modeled from a promiscuous Th epitope of the F protein of measles virus (Partidos et al. 1991). The model Th epitope, shown in Table 1 as “MVF Th (UBITh® 5)” (SEQ ID NO: 6) corresponds to residues 288-302 of the measles virus F protein. MVF Th (SEQ ID NO: 6) was modified to the SSAL1 Th1 prototype (SEQ ID NOs: 1-4) by adding a charged residue Glu/Asp at position 1 to increase the charge surrounding the hydrophobic face of the epitope; adding or retaining a charged residues or Gly at positions 4, 6, 12 and 14; and adding or retaining a charged residue or Gly at positions 7 and 11 in accordance with the “Rothbard Rule”. The hydrophobic face of the Th epitope comprise residues at positions 2, 5, 8, 9, 10, 13, and 16. Hydrophobic residues commonly associated with promiscuous epitopes were substituted at these positions to provide the combinatorial Th SSAL epitopes, SSAL1 Th1 (SEQ ID NOs: 1-4). Another significant feature of the prototype SSAL1 Th1 (SEQ ID NOs: 1-4) is that positions 1 and 4 is imperfectly repeated as a palindrome on either side of position 9, to mimic an MHC-binding motif. This “1, 4, 9” palindromic pattern of SSAL1 Th1 was further modified in SEQ ID NO: 2 (Table 1) to more closely reflect the sequence of the original MvF model Th (SEQ ID NO: 6).

Combinatorial artificial Th epitopes can be simplified to provide a series of single-sequence epitopes. For example, the combinatorial sequence of SEQ ID NO: 5 can be simplified to the single sequence Th epitopes represented by SEQ ID NOs: 1-4. These single sequence Th epitopes can be coupled to target antigenic sites to provide enhanced immunogenicity.

In some embodiments, the immunogenicity of the Th epitopes may be improved by extending the N terminus with a non-polar and a polar uncharged amino acid, e.g., Ile and Ser, and extending the C terminus by a charged and hydrophobic amino acid, e.g., Lys and Phe. In addition, the addition of a Lysine residue or multiple lysine residues (e.g., KKK) to the Th epitopes can improve the solubility of the peptide in water. Further modifications included the substitution of the C-termini by a common MHC-binding motif AxTxIL (Meister et al, 1995).

An artificial Th epitope can be a known natural Th epitope or an SSAL peptide prototype. In some embodiments, a Th epitope from an SSAL can incorporate combinatorial MHC molecule binding motifs intended to elicit broad immune responses among the members of a genetically diverse population. In some embodiments, a SSAL peptide prototype can be designed based on Th epitopes of the measles virus and hepatitis B virus antigens, modified by introducing multiple MHC-binding motifs. In some embodiments, an artificial Th epitope can simplify, add, or and/or modify multiple MHC-binding motifs to produce a series of novel artificial Th epitopes. In some embodiments, newly adapted promiscuous artificial Th sites can be incorporated into synthetic peptide immunogens bearing a variety of target antigenic sites. In some embodiments, resulting chimeric peptides can stimulate effective antibody responses to target antigenic sites.

Artificial Th epitopes of the disclosure can be contiguous sequences of natural or non-natural amino acids that comprise a class II MHC molecule binding site. In some embodiments, an artificial Th epitope can enhance or stimulate an antibody response to a target antigenic site. In some embodiments, a Th epitope can consist of continuous or discontinuous amino acid segments. In some embodiments, not every amino acid of a Th epitope is involved with MHC recognition. In some embodiments, the Th epitopes of the invention can comprise immunologically functional homologues, such as immune-enhancing homologues, cross reactive homologues, and segments thereof. In some embodiments, functional Th homologues can further comprise conservative substitutions, additions, deletions, and insertions of 1, 2, 3, 4, 5, 6, 7, 8, 9, or 10 amino acid residues and provide the Th-stimulating function of a Th epitope.

Th epitopes can be attached directly to the target site. In some embodiments, the Th epitopes can be attached to the target site through an optional heterologous spacer, e.g., a peptide spacer such as Gly-Gly or (ε-N)Lys. The spacer physically separates the Th epitope from the B cell epitope, and can disrupt the formation of any artificial secondary structures created by the linking of the Th epitope or a functional homologue with the target antigenic site, thereby eliminating any interference with the Th and/or B cell responses.

Th epitopes include idealized artificial Th epitopes and combinatorial idealized artificial Th epitopes, as shown in Table 1. In some embodiments, the Th epitope is a promiscuous Th cell epitope of SEQ ID NOs: 1-52, any homologue thereof, and/or any immunological analogue thereof. Th epitopes also include immunological analogues of Th epitopes. Immunological Th analogues include immune-enhancing analogs, cross-reactive analogues and segments of any of these Th epitopes that are sufficient to enhance or stimulate an immune response to the target antigenic site.

Functional immunologically analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the target antigenic site. Table 1 identifies another variation of a functional analogue for Th epitope peptide. In particular, SEQ ID NOs: 6 and 7 of MvF1 and MvF2 Th are functional analogues of SEQ ID NOs: 16 and 17 of MvF4 and MvF5 in that they differ in the amino acid frame by the deletion (SEQ ID NOs: 6 and 7) or the inclusion (SEQ ID NOs: 16 and 17) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 6-18) and from Hepatitis Surface protein HBsAg 1-3 Ths (SEQ ID NOs: 19-31).

The Th epitope in peptide immunogen construct can be covalently linked at either N- or C-terminal end of the target antigenic site to produce a chimeric Th/B cell site peptide immunogen. In some embodiments, a Th epitope can be covalently attached to the target antigenic site via chemical coupling or via direct synthesis. In some embodiments, the Th epitope is covalently linked to the N-terminal end of the target antigenic site. In other embodiments, the Th epitope is covalently linked to the C-terminal end of the target antigenic site. In certain embodiments, more than one Th epitope is covalently linked to the target antigenic site. When more than one Th epitope is linked to the target antigenic site, each Th epitope can have the same amino acid sequence or different amino acid sequences. In addition, when more than one Th epitope is linked to the target antigenic site, the Th epitopes can be arranged in any order. For example, the Th epitopes can be consecutively linked to the N-terminal end of the target antigenic site, or consecutively linked to the C-terminal end of the target antigenic site, or a Th epitope can be covalently linked to the N-terminal end of the target antigenic site while a separate Th epitope is covalently linked to the C-terminal end of the target antigenic site. There is no limitation in the arrangement of the Th epitopes in relation to the target antigenic site.

In some embodiments, the Th epitope is covalently linked to the target antigenic site directly. In other embodiments, the Th epitope is covalently linked to the target antigenic site through a heterologous spacer described in further detail below.

### Methods of Synthesis

The peptide immunogens of the disclosure can be synthesized using chemical methods. In some embodiments, the peptide immunogens of the disclosure can be synthesized using solid phase peptide synthesis. In some embodiments, the peptides of the invention are synthesized using automated Merrifield solid phase peptide synthesis using t-Boc or Fmoc to protect α-NH₂ or side chain amino acids.

The heterologous Th epitope peptides presented as a combinatorial sequence contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 5, 10, 13, 16, 24, and 27 which are shown in Table 1. Th epitope peptides of the present invention provide broad reactivity and immunogenicity to animals and patients from genetically diverse populations.

Interestingly, inconsistencies and/or errors that might be introduced during the synthesis of the Th epitope, B cell epitope, and/or the peptide immunogen construct containing a Th epitope and B cell epitope most often do not hinder or prevent a desired immune response in a treated animal. In fact, inconsistencies/errors that might be introduced during the peptide synthesis generate multiple peptide analogues along with the targeted peptide syntheses. These analogues can include amino acid insertion, deletion, substitution, and premature termination. As described above, such peptide analogues are suitable in peptide preparations as contributors to antigenicity and immunogenicity when used in immunological application either as solid phase antigen for purpose of immunodiagnosis or as immunogens for purpose of vaccination.

Peptide immunogen constructs comprising Th epitopes are produced simultaneously in a single solid-phase peptide synthesis in tandem with the target antigenic site. Th epitopes also include immunological analogues of Th epitopes. Immunological Th analogues include immune-enhancing analogs, cross-reactive analogues and segments of any of these Th epitopes that are sufficient to enhance or stimulate an immune response to the target antigenic site.

After the complete assembly of a desired peptide immunogen, the solid phase resin can be treated to cleave the peptide from the resin and to remove the functional groups on the amino acid side chains. The free peptide can be purified by HPLC and characterized biochemically. In some embodiments, the free peptides are characterized biochemically using amino acid analysis. In some embodiments, the free peptides are characterized using peptide sequence. In some embodiments, the free peptides are characterized using mass spectrometry.

The peptide immunogens of the invention can be synthesized using haloacetylated and cysteinylated peptides through the formation of a thioether linkage. In some embodiments, a cysteine can be added to the C terminus of a Th-containing peptide, and the thiol group of the cysteine residue can be used to form a covalent bond to an electrophilic group such as a N^{α} chloroacetyl-modified group or a maleimide-derivatized α- or ε-NH₂ group of a lysine residue. The resulting synthetic intermediate can be attached to the N-terminus of a target antigenic site peptide.

Longer synthetic peptide conjugates can be synthesized using nucleic acid cloning techniques. In some embodiments, the Th epitopes of the invention can be synthesized by expressing recombinant DNA and RNA. To construct a gene expressing a Th/target antigenic site peptide of this invention, an amino acid sequence can be reverse translated into a nucleic acid sequence. In some embodiments, an amino acid sequence is reverse translated into a nucleic acid sequence using optimized codons for the organism in which the gene will be expressed. A gene encoding the peptide can be made. In some embodiments, a gene encoding a peptide can be made by synthesizing overlapping oligonucleotides that encode the peptide and necessary regulatory elements. The synthetic gene can be assembled and inserted into a desired expression vector.

The synthetic nucleic acid sequences of the disclosure can include nucleic acid sequences that encode Th epitopes of the invention, peptides comprising Th epitopes, immunologically functional homologues thereof, and nucleic acid constructs characterized by changes in the non-coding sequences that do not alter the immunogenic properties of the peptide or encoded Th epitope. The synthetic gene can be inserted into a suitable cloning vector, and recombinants can be obtained and characterized. The Th epitopes and peptides comprising the Th epitopes can then be expressed under conditions appropriate for a selected expression system and host. The Th epitope or peptide can be purified and characterized.

### Pharmaceutical Compositions

The present disclosure also describes pharmaceutical compositions comprising peptide immunogens of the disclosure. In some embodiments, a pharmaceutical composition of the disclosure can be used as a pharmaceutically acceptable delivery system for the administration of peptide immunogens. In some embodiments, a pharmaceutical composition of the disclosure can comprise an immunologically effective amount of one or more of the peptide immunogens.

The peptide immunogens of the invention can be formulated as immunogenic compositions. In some embodiments, an immunogenic composition can comprise adjuvants, emulsifiers, pharmaceutically-acceptable carriers or other ingredients routinely provided in vaccine compositions. Adjuvants or emulsifiers that can be used in this invention include alum, incomplete Freund's adjuvant (IFA), liposyn, saponin, squalene, L121, emulsigen, monophosphoryl lipid A (MPL), dimethyldioctadecylammonium bromide (DDA), QS21, and ISA 720, ISA 51, ISA 35, ISA 206, and other efficacious adjuvants and emulsifiers. In some embodiments, a composition of the invention can be formulated for immediate release. In some embodiments, a composition of the invention can be formulated for sustained release.

Adjuvants used in the pharmaceutical composition can include oils, aluminum salts, virosomes, aluminum phosphate (e.g. ADJU-PHOS®), aluminum hydroxide (e.g. ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains Montanide™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e. w/o/w) emulsion with EMULSIGEN or EMULSIGEN D as the adjuvant.

In some embodiments, a composition is formulated for use as a vaccine. A vaccine composition can be administered by any convenient route, including subcutaneous, oral, intramuscular, intraperitoneal, parenteral, or enteral administration. In some embodiments, the immunogens are administered in a single dose. In some embodiments, immunogens are administered over multiple doses.

Pharmaceutical compositions can be prepared as injectables, either as liquid solutions or suspensions. Liquid vehicles containing the tau peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

The composition of the instant invention can contain an effective amount of one or more peptide immunogens and a pharmaceutically acceptable carrier. In some embodiments, a composition in a suitable dosage unit form can contain about 0.5 μg to about 1 mg of a peptide immunogen per kg body weight of a subject. In some embodiments, a composition in a suitable dosage unit form can contain about 10 μg, about 20 μg, about 30 μg, about 40 μg, about 50 μg, about 60 μg, about 70 μg, about 80 μg, about 90 μg, about 100 μg, about 200 μg, about 300 μg, about 400 μg, about 500 μg, about 600 μg, about 700 μg, about 800 μg, about 900 μg, or about 1000 μg of a peptide immunogen per kg body weight of a subject. In some embodiments, a composition in a suitable dosage form can contain about 100 μg, about 150 μg, about 200 μg, about 250 μg, about 300 μg, about 350 μg, about 400 μg, about 450 μg, or about 500 μg of a peptide immunogen per kg body weight of a subject. In some embodiments, a composition in a suitable dosage unit form can contain about 0.5 μg to about 1 mg of a peptide immunogen per kg body weight of a subject. In some embodiments, a composition in a suitable dosage unit form can contain about 10 μg, about 20 μg, about 30 μg, about 40 μg, about 50 μg, about 60 μg, about 70 μg, about 80 μg, about 90 μg, about 100 μg, about 200 μg, about 300 μg, about 400 μg, about 500 μg, about 600 μg, about 700 μg, about 800 μg, about 900 μg, or about 1000 μg of a peptide immunogen. In some embodiments, a composition in a suitable dosage form can contain about 100 μg, about 150 μg, about 200 μg, about 250 μg, about 300 μg, about 350 μg, about 400 μg, about 450 μg, or about 500 μg of a peptide immunogen.

When delivered in multiple doses, a composition can be divided into an appropriate amount per dose. In some embodiments, a dose is about 0.2 mg to about 2.5 mg. In some embodiments, a dose is about 1 mg. In some embodiments, a dose is about 1 mg and is administered by injection. In some embodiments, a dose is about 1 mg and is administered intramuscularly. In some embodiments, a dose can be followed by a repeat (booster) dose. Dosages can be optimized depending on the age, weight, and general health of the subject.

Vaccines comprising mixtures of peptide immunogens can provide enhanced immunoefficacy in a broader population. In some embodiments, a mixture of peptide immunogens comprises Th sites derived from MVF Th and HBsAg Th. In some embodiments, vaccines comprising mixtures of peptide immunogens can provide an improved immune response to the target antigenic site.

The immune response to Th/target antigenic site conjugates can be improved by delivery through entrapment in or on biodegradable microparticles. In some embodiments, peptide immunogens can be encapsulated with or without an adjuvant, and such microparticles can carry an immune stimulatory adjuvant. In some embodiments, microparticles can be co-administered with peptide immunogens to potentiate immune responses.

### Immunostimulatory Complexes

The present disclosure is also directed to pharmaceutical compositions containing an tau peptide immunogen construct in the form of an immunostimulatory complex with a CpG oligonucleotide. Exemplary CpG oligonucleotides are shown in Table 5. Such immunostimulatory complexes are specifically adapted to act as an adjuvant and as a peptide immunogen stabilizer. The immunostimulatory complexes are in the form of a particulate, which can efficiently present the tau peptide immunogen to the cells of the immune system to produce an immune response. The immunostimulatory complexes may be formulated as a suspension for parenteral administration. The immunostimulatory complexes may also be formulated in the form of w/o emulsions, as a suspension in combination with a mineral salt or with an in-situ gelling polymer for the efficient delivery of the tau peptide immunogen to the cells of the immune system of a host following parenteral administration.

The stabilized immunostimulatory complex can be formed by complexing an tau peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the tau peptide immunogen construct is designed to contain a cationic portion that is positively charged at a pH in the range of 5.0 to 8.0. The net charge on the cationic portion of the tau peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the tau peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the tau peptide immunogen construct is the heterologous spacer. In certain embodiments, the cationic portion of the tau peptide immunogen construct has a charge of +4 when the spacer sequence is (a, ε-N)Lys or ε-N-Lys-Lys-Lys-Lys (SEQ ID NO: 53).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X1 is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

### Applications

The peptides of the invention can be useful in medical and veterinary applications. In some embodiments, the peptides of the invention can be used as vaccines to provide protective immunity from infectious disease, immunotherapies for treating disorders resulting from malfunctioning normal physiological processes, immunotherapies for treating cancer, and as agents to intervene or modify normal physiological processes.

The artificial Th epitopes of the disclosure can provoke an immune response when combined with target B cell epitopes of various microorganisms, proteins, or peptides. In some embodiments, an artificial Th epitopes of the disclosure can be linked to one target antigenic site. In some embodiments, an artificial Th epitope of the disclosure can be linked to two target antigenic sites.

The artificial Th epitopes of the disclosure can be linked to target antigenic sites to prevent and/or treat various diseases and conditions. In some embodiments, a composition of the invention can be used for the prevention and/or treatment of neurodegenerative diseases, infectious diseases, arteriosclerosis, prostate cancer, prevention of boar taint, immunocastration of animals, the treatment of endometriosis, breast cancer and other gynecological cancers affected by the gonadal steroid hormones, and for contraception in males and females. For example, the artificial Th epitopes can be linked to the antigenic sites of the following proteins:
a. Somatostatin to promote growth in farm animals.
b. IgE to treat allergic diseases.
c. The CD4 receptor of Th cells to treat and/or prevent human immunodeficiency virus (HIV) infections and immune disorders.
d. Foot-and-mouth disease (FMD) virus capsid protein to prevent FMD.
e. HIV virion epitopes to prevent and treat HIV infections.
f. The circumsporozoite antigen of Plasmodium falciparum to prevent and treat malaria.
g. CETP to prevent and treat arteriosclerosis.
h. Aβ to treat or vaccinate against Alzheimer's disease.
i. Alpha-synuclein to treat or vaccinate against Parkinson's disease.
j. Tau to treat and vaccinate against tauopathies including Alzheimer's disease.
k. IL-31 to treat atopic dermatitis.

The use of heterologous artificial Th epitopes has been found to be particularly important for targeting proteins involved in neurodegenerative diseases (e.g., Aβ, alpha-synuclein, Tau). Specifically, peptide immunogens that contain endogenous Th epitopes of targeted neurodegenerative proteins can cause inflammation of the brain when administered to a subject. In contrast, peptide immunogen constructs that contain a heterologous artificial Th epitope liked to an antigenic site of a neurodegenerative protein does not cause brain inflammation.

### Amyloid β

The Aβ peptide is thought to be the pivot for the onset and progression of Alzheimer's disease. Toxic forms of Aβ oligomers and Aβ fibrils are suggested to be responsible for the death of synapses and neurons that lead to the pathology of Alzheimer's disease and dementia. A successful disease-modifying therapy for Alzheimer's disease can include products that affect the disposition of Aβ in the brain.

A peptide immunogen of the disclosure can comprise Th cell epitopes and Aβ-targeting peptides. In some embodiments, the Th cell epitope is Th1 or Th2. In some embodiments, the peptide immunogen can comprise Th1 and Th2. The Aβ-targeting peptide, or B cell epitopes, can be Aβ₁₋₁₄, Aβ₁₋₁₆, Aβ₁₋₂₈, Aβ₁₇₋₄₂, or Aβ₁₋₄₂. In some embodiments, the Aβ-targeting peptide is Aβ₁₋₁₄. As used herein, the term Aβ_{x-y} indicates an Aβ sequence from amino acid x to amino acid y of the full-length wild-type Aβ protein.

A peptide immunogen of the disclosure can comprise more than one Aβ-targeting peptide. In some embodiments, a peptide immunogen can comprise two Aβ-targeting peptides. In some embodiments, a peptide immunogen can comprise one Aβ₁₋₁₄ and one Aβ₁₋₄₂ peptide. In some embodiments, a peptide immunogen can comprise two Aβ₁₋₁₄-targeting peptides. In some embodiments, a peptide immunogen can comprise two Aβ₁₋₁₄-targeting peptides, each linked to different Th cell epitopes as a chimeric peptide.

The present disclosure also provides Aβ₁₋₁₄ peptide vaccines comprising two Aβ₁₋₁₄-targeting peptides, each linked to different Th cell epitopes as a chimeric peptide. In some embodiments, a chimeric Aβ₁₋₁₄ peptide can be formulated in a Th1-biased delivery system to minimize T-cell inflammatory reactivity. In some embodiments, a chimeric Aβ₁₋₁₄ peptide can be formulated in a Th2-biased delivery system to minimize T-cell inflammatory reactivity.

### General

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Example 1

### Preparation of Peptides and Peptide Immunogen Constructs

Peptides, including peptide immunogen constructs, were synthesized using automated solid-phase synthesis, purified by preparative HPLC, and characterized by matrix-assisted laser desorption ionization-time of flight (MALDI-TOF) mass spectrometry, amino acid analysis, and reverse-phase HPLC.

The Aβ vaccine (UB-311) comprises two peptide immunogens, each with an N-terminal Aβ₁₋₁₄ peptide, synthetically linked through an amino acid spacer to different Th cell epitope peptides (UBITh® epitopes) derived from two pathogen proteins: hepatitis B surface antigen and measles virus fusion protein. Specifically, the peptide immunogen linked to a measles virus fusion protein was Aβ₁₋₁₄-εK-KKK-MvF5 Th (SEQ ID NO: 67) and the peptide immunogen linked to a hepatitis B surface antigen was Aβ₁₋₁₄-εK-HBsAg3 Th (SEQ ID NO: 68).

UB-311 was formulated in an alum-containing Th2-biased delivery system and contained the peptides Aβ₁₋₁₄-εK-HBsAg3 and Aβ₁₋₁₄-εK-KKK-MvF5 Th in an equimolar ratio. The two Aβ immunogens were mixed with polyanionic CpG oligodeoxynucleotide (ODN) to form stable immunostimulatory complexes of micron-sized particulates. An aluminum mineral salt (ADJU-PHOS®) was added to the final formulation, along with sodium chloride for tonicity and 0.25% 2-phenoxyethanol as a preservative.

The sequences of several exemplary target antigenic sites (B cell epitopes) are shown in Table 3. The sequences of several exemplary peptide immunogen constructs containing Aβ₁₋₁₄ as the target antigenic site covalently linked to a Th epitope are shown in Table 4.

### Example 2

### Exclusive Immunogenicity of Peptide Immunogen Constructs in Guinea Pigs that Targets AB Peptides but not TH Epitopes

Six guinea pigs were immunized at Weeks 0 and 4 with peptide immunogen constructs Aβ₁₋₁₄-EK-KKK-MvF5 (SEQ ID NO: 67) and Aβ₁₋₁₄-EK-HBsAg3 (SEQ ID NO: 68) formulated together in equimolar ratio. At Week 8, animals were bled and serum samples were collected to determine anti-Aβ peptide and anti-Th epitope antibody titers (log₁₀) by ELISA test. The antibody response of all 6 guinea pigs specifically targeted the Aβ₁₋₄₂ peptide and not the two artificial Th epitopes (MvF5 Th and HBsAg3 Th), as shown in Table 6.

### Example 3

### Cellular Immune Response in Baboons and Macaque Peripheral Blood Mononuclear Cell (PBMC) Cultures

Peripheral blood mononuclear cells (PBMC) from baboons and from Cynomolgus macaques were isolated by Ficoll-hypaque gradient centrifugation. For peptide-induced proliferation and cytokine production, cells (2×10⁵ per well) were cultured alone or with individual peptide domains added (including, Aβ₁₋₁₄, Aβ₁₋₄₂, UBITh®, and non-relevant peptide). Mitogens (PHA, PWM, Con A) were used as positive controls (10 μg/mL at 1% v/v of culture). On day 6, 1 μCi of 3H-thymidine (3H-TdR) was added to each of three replicate culture wells. After 18 h of incubation, cells were harvested and 3H-TdR incorporation was determined. The stimulation index (S.I.) represents the cpm in the presence of antigen divided by the cpm in the absence of antigen; a S.I.>3.0 was considered significant.

Cytokine analyses (IL2, IL6, IL10, IL13, TNFα, IFNγ) from the Cynomolgus macaque PBMC cultures were performed on aliquots of culture medium alone or in the presence of peptide domains or mitogens. Monkey-specific cytokine sandwich ELISA kits (U-CyTech Biosciences, Utrecht, The Netherlands) were used to determine the concentration of individual cytokines following kit instructions.

PBMCs were isolated from whole blood collected from macaques at 15, 21, and 25.5 weeks. The isolated PBMCs were cultured in the presence of various Aβ peptides (Aβ₁₋₁₄ and Aβ₁₋₄₂).

No proliferation responses by lymphocytes were observed when Aβ₁₋₁₄ peptide was added to the culture medium. However, positive proliferation responses were found when the Aβ₁₋₄₂ peptide was added to the PBMC cultures.

The PBMC samples collected at 15, 21 and 25.5 weeks were also tested for cytokine secretion in the presence of Aβ peptides or PHA mitogen. As shown in Table 7, three cytokines (IL2, IL6, TNFα) showed detectable secretion in response to the full-length Aβ₁₋₄₂ peptide but not to the Aβ₁₋₁₄ peptide; up-regulation of cytokine secretion was not detected in the UBITh® AD vaccine-treated samples when compared to the placebo vaccine samples. Three other cytokines (IL-10, IL-13, IFNγ) tested in the presence of the Aβ peptides were below the assay detection limit in all PBMC cultures.

The macaques were immunized with the UB-311 vaccine having only the N-terminal Ai-14 peptide immunogens with foreign T helper epitopes, without the Aβ₁₇₋₄₂ peptide domain, indicating that the positive proliferation results noted in the PBMC cultures in the presence of Aβ₁₋₄₂ peptide were not related to the UB-311 vaccine response, but rather were a background response to native AD.

These results support the safety of the UB-311 vaccine that has only Aβ₁₋₁₄ and foreign T helper epitopes, showing that it does not generate potentially inflammatory anti-self cell-mediated immune responses to Aβ peptides in the normal macaques. In contrast, the adverse events associated with encephalitis in the clinical trial studies of the AN-1792 vaccine were attributed in part, to the inclusion of T cell epitopes within the fibrillar/aggregated Aβ₁₋₄₂ immunogen of that vaccine.

### Example 4

### Lymphocyte Proliferation Analysis and Cytokine Analysis

Peripheral blood mononuclear cells (PBMC) from patients with Alzheimer's Disease were isolated by Ficoll-hypaque gradient centrifugation. For peptide-induced proliferation and cytokine production, cells (2.5×10⁵ per well) were cultured in triplicate alone or with individual peptide domains added (at a final concentration of 10 μg/mL), including Aβ₁₋₁₄ (SEQ ID NO: 56), Aβ₁₋₁₆ (SEQ ID NO: 57), Aβ₁₋₂₈ (SEQ ID NO: 59), Aβ₁₇₋₄₂ (SEQ ID NO: 58), Aβ₁₋₄₂ (SEQ ID NO: 60) and a non-relevant 38-mer peptide (p1412). Cultures were incubated at 37° C. with 5% CO₂ for 72 hours, and then 100 μL of supernatant was removed from each well and frozen at −70° C. for cytokine analysis. Ten μL of culture medium containing 0.5 μCi of ³H-thymidine (³H-TdR, Amersham, Cat No. TRK637) was added to each well and incubated for 18 hr, followed by detection of radioisotope incorporation by liquid scintillation counting. The mitogen phytohemagglutinin (PHA) was used as a positive control for lymphocyte proliferation. Cells cultured alone without Aβ peptide or PHA mitogen were used as the negative and positive controls. The stimulation index (SI) was calculated as mean counts per min (cpm) of triplicate experimental cultures with Aβ peptide divided by mean cpm of triplicate negative control cultures; a SI>3.0 was considered a significant proliferation response.

a. Proliferation Analysis

Peripheral blood mononuclear cell samples were isolated from whole blood collected at week 0 (baseline) and week 16 (4 weeks after the third dose) and then cultured in the absence or presence of various Aβ peptides. As shown in Table 8, no significant proliferation response by lymphocytes was observed when Aβ₁₋₁₄, other Aβ peptides, or p1412 (a non-relevant control peptide) were added to the culture medium. As expected, positive proliferation responses were noted when PHA mitogen was added to culture medium. The observation of similar responses to PHA before and after UB 311 immunization (p=0.87) suggests no significant alteration in study subjects' immune functions (Table 8).

Statistical Analysis. The differences in lymphocyte proliferation between Week 0 and Week 16 were examined by the paired t-test. Statistical significance levels were determined by 2-tailed tests (p<0.05). R version 2.14.1 was used for all statistical analyses.

b. Cytokine Analysis

Cytokine analyses (IL-2, IL-6, IL-10, TNF-α, IFN-γ) from the PBMC cultures were performed on aliquots of culture medium with cells alone or in the presence of Aβ peptide domains or PHA. Human-specific cytokine sandwich ELISA kits (U-CyTech Biosciences, Utrecht, The Netherlands) were used to determine the concentrations (pg/mL) of individual cytokines following the manufacturer's instructions (Clin Diag Lab Immunol. 5(1):78-81 (1998)).

The PBMC samples collected at week 0 and week 16 were also tested for cytokine secretion either with cells alone (negative control) or in the presence of Aβ peptides, p1412 (non-relevant peptide) or PHA mitogen (positive control) after being cultured for 3 days. The quantifiable range of the kit is between 5 and 320 μg/mL. Any measured concentration below 5 μg/mL or above 320 μg/mL was indicated as below quantification limit (BQL) or above quantification limit (AQL), respectively. However, for statistical considerations, BQL or AQL was replaced with the lower (5 μg/mL) or upper (320 μg/mL) quantifiable limit, respectively. The mean concentrations of each cytokine at week 0 and week 16 are shown in Table 9. As expected, there were significant increases in cytokine production in the presence of PHA, the positive control, except for IL-2. The production of cytokines in response to the stimulation with Aβ₁₋₁₄, or other Aβ peptides was observed at baseline (week 0) and week 16, but most values appeared similar to the corresponding negative controls (cells alone).

In order to assess the change of cell-mediated immune response after immunization, the change of mean cytokine concentrations from baseline to week 16 was compared with that of the negative controls and examined by paired Wilcoxon signed-rank test. Four cytokines (IFN-γ, IL-6, IL-10, TNF-α) showed notable increase in secretion in response to full-length Aβ₁₋₄₂ peptide; this observation may be due to the conformational epitopes of Aβ₁₋₄₂ aggregates. Up-regulation of cytokine secretion was not detected in Aβ₁₋₁₄ or other Aβ peptides.

c. Summary

UB-311 vaccine contains two peptide immunogens each with a N-terminal Aβ₁₋₁₄ peptide synthetically linked to MvF5 Th and HBsAg3 Th epitopes respectively. In vitro lymphocyte proliferation and cytokine analysis were used to evaluate the impact of immunization of UB-311 vaccine on the cellular immune response. No proliferation responses by lymphocytes were observed when the Aβ₁₋₁₄ peptide or any other Aβ peptides was added to culture medium as shown in Table 8. Up-regulation of cytokine secretion by lymphocytes of UB-311 vaccine-immunized patients was not detected upon treatment with the Aβ₁₋₁₄ and other Aβ peptides except for Aβ₁₋₄₂, which elicited appreciable increase of four cytokines (IFN-γ, IL-6, IL-10, TNF-α) after UB-311 immunization at week 16 when compared to week 0 levels before treatment (Table 9). The increase of cytokine release through Th2 type T cell response is more likely unrelated to the UB-311 vaccine response since no up-regulation detected with Aβ₁₋₁₄ alone. The response to Aβ₁₋₄₂ is suspected to be a background response to native Aβ that may be related to native T helper epitopes identified on AD 1-42. The lack of IL-2 production in response to PHA was observed, which is consistent with the findings reported by Katial R K, et al. in Clin Diagn Lab Immunol 1998; 5:78-81, under similar experimental conditions with normal human PBMC. In conclusion, these results showed that the UB-311 vaccine did not generate potentially inflammatory anti-self, cell-mediated immune responses in patients with mild to moderate Alzheimer's disease who participated in the phase I clinical trial, thus further demonstrating the safety of the UB-311 vaccine.

### Example 5

### Promiscuous Artificial Th Responsive Cells can be Detected in Naïve Peripheral Blood Mononuclear Cells (PMBC) in Normal Blood Donors with Moderate Immunogenic Inflammatory Response when Compared to Negative Control

ELISpot Assay was employed to detect promiscuous artificial Th responsive cells in naïve peripheral blood mononuclear cells in normal blood donors to assess their potency to elicit inflammatory responses when compared to a potent mitogen Phytohemagglutinin (PHA) and negative control.

ELISpot assays employ the sandwich enzyme-linked immunosorbent assay (ELISA) technique. For detection of T cell activation, IFN-γ or related cytokine was detected as an analyte. Either a monoclonal or polyclonal antibody specific for the chosen analyte was pre-coated onto a PVDF (polyvinylidene difluoride)-backed microplate. Appropriately stimulated cells were pipetted into the wells and the microplate was placed into a humidified 37° C. C02 incubator for a specified period of time. During this incubation period, the immobilized antibody, in the immediate vicinity of the secreting cells, bound to secreted analyte. After washing away any cells and unbound substances, a biotinylated polyclonal antibody specific for the chosen analyte was added to the wells. Following a wash to remove any unbound biotinylated antibody, alkaline-phosphatase conjugated to streptavidin was added. Unbound enzyme was subsequently removed by washing and a substrate solution (BCIP/NBT) was added. A blue-black colored precipitate formed and appeared as spots at the sites of cytokine localization, with each individual spot representing an individual analyte-secreting cell. The spots were counted with an automated ELISpot reader system or manually, using a stereomicroscope.

In the in vitro study conducted, PHA at 10 μg/mL culture was used as a positive control. UBITh® 1 (SEQ ID NO:17) and UBITh® 5 (SEQ ID NO:6) peptides were tested for the number of responsive cells present in the peripheral blood mononuclear cells in regular normal blood donors. A mixture of promiscuous artificial Th epitope peptides with SEQ ID NOs: 33 to 52 were prepared as another positive control. Media alone was used as the negative control in a standard T cell stimulation cell culture condition. Briefly, 100 μL/well of PBMCs (2×10⁵ cells) stimulated with mitogen (PHA at 10 μg/mL), or Th antigen (UBITh® 1, UBITh® 5 or mixture of multi-Ths at 10 μg/mL) were incubated at 37° C. in a CO₂ incubator for 48 hours. The supernatant from wells/plates were collected. The cells on the plates were washed and processed for detection of the target analyte, IFN-γ.

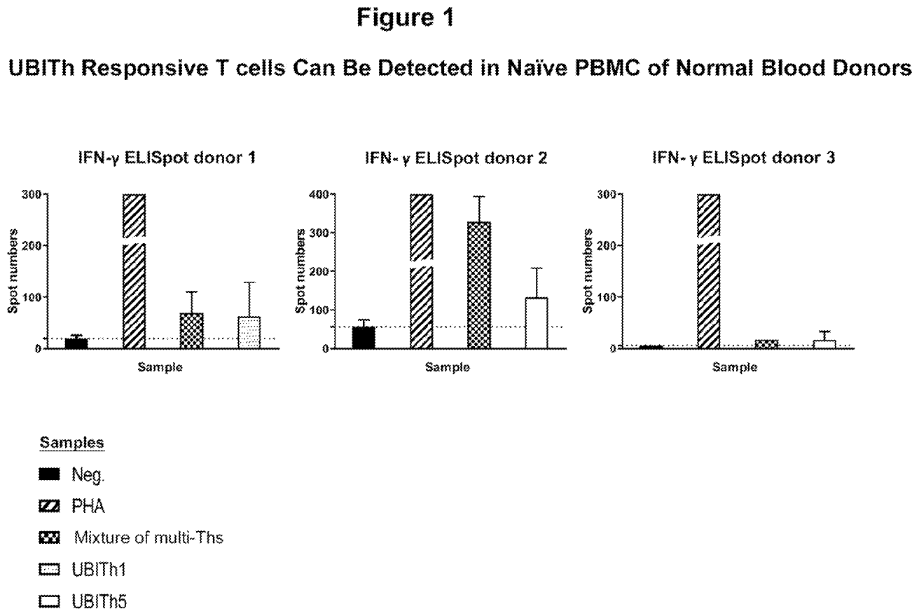

As shown in FIG. 1, representative donors 1, 2, and 3 were tested for their responsive cells to promiscuous artificial UBITh® 1 or UBITh® 5 epitope peptides. An overwhelming IFN-γ ELISPOT number was always detected with naïve donor (PBMCs cultivated with PHA; too numerous to count) while PBMCs cultivated with control media gave a background IFN-γ ELISPOT number between 5 to 50. Moderate ELISPOT numbers were detected for naïve donor PBMCs cultivated with UBITh® 1 or UBITh® 5 from 20 to around 120. A mixture of multi Th peptides with SEQ ID NOs: 33-52 was also cultivated with naïve donor PBMCs for comparison with ELISPOT numbers come in from 20 to about 300 as expected. Such stimulatory responses triggered by the UBITh® 1 or UBITh® 5 peptides are about 3 to 5 times compared to the negative controls.

In summary, promiscuous artificial Th responsive cells can be readily detected in naïve donor PBMCs which stand ready to mount immune responses to help the B cell antibody production and the corresponding effector T cell responses by secreting signature cytokines. IFN-γ was used as one example here to illustrate this stimulatory nature of these Th epitope peptides. However, such stimulatory inflammatory responses are moderate enough to mount a suitable effector cell responses (B cell for antibody production, cytotoxic T cells for killing of target antigenic cells) so as not to cause untoward inflammatory pathophysiological responses during a vaccination process.

TABLE 1
Amino Acid Sequences of Pathogen
Protein Derived Th Epitopes
Including Idealized Artificial
Th Epitopes for Employment in
the Design of Peptide Immunogen
Constructs

| | | SEQ |
|---|---|---|
| | | ID |
| Description | Sequence | NO |
| MvF Th | DLSDLKGLLLHKLDGL | 1 |
| (SSAL1 Th1) | EI EIR III RIE I | 2 |
| | V V VVV V V | 3 |
| | F F FFF F F | 4 |
| | XXSXXXGXXXHXXXGX | 5 |
| MvF1 Th | LSEIKGVIVHRLEGV | 6 |
| (UBITh®5) | | |
| MvF2 Th | ISEIKGVIVHKIEGI | 7 |
| | ISISEIKGVIVHKIEGILF | 8 |
| MvF3 Th | T RT TR T | 9 |
| | ISIXEIXXVIVXXIEXILF | 10 |
| KKKMvF3 Th | KKKISISEIKGVIVHKIEGILF | 11 |
| | T RT TR T | 12 |
| | KKKISIXEIXXVIVXXIEXILF | 13 |
| | ISISEIKGVIVHKIETILF | 14 |
| MvF4 Th | T RT TR | 15 |
| (UBITh®3) | ISIXEIXXVIVXXIETILF | 16 |
| MvF5 Th | ISITEIKGVIVHRIETILF | 17 |
| (UBITh®1) | | |
| KKKMvF5 Th | KKKISITEIKGVIVHRIETILF | 18 |
| (UBITh®1a) | | |
| HBsAgl Th | KKKLFLLTKLLTLPQSLD | 19 |
| (SSAL2 Th2) | RRRIKII RII I L IR | 20 |
| | VRVV VVV I V | 21 |
| | F FF FFF V F | 22 |
| | F | 23 |
| | XXXXXXXTXXXTXPXSXX | 24 |
| HBsAg2 Th | KKKIITITRIITIPQSLD | 25 |
| | FFLL L ITTI | 26 |
| | KKKXXXXTRIXTIXXXXD | 27 |
| HBsAg3 Th | KKKIITITRIITIITTID | 28 |
| (UBITh®2) | | |
| HBsAg Th | FFLLTRILTIPQSLD | 29 |
| (UBITh®4) | | |
| KKK-HBsAg Th | KKKFFLLTRILTIPQSLD | 30 |
| HBsAg Th | FFLLTRILTIPQSL | 31 |
| Bordetella | GAYARCPNGTRALTVAELRGNAEL | 32 |
| pertussis Th | | |
| (UBITh®7) | | |
| Cholera | ALNIWDRFDVFCTLGATTGYLKGNS | 33 |
| Toxin Th | | |
| Clostridium | QYIKANSKFIGITEL | 34 |
| tetani TT1 Th | | |
| Clostridium | KKQYIKANSKFIGITEL | 35 |
| tetani1 Th | | |
| (UBITh®6) | | |
| Clostridium | FNNFTVSFWLRVPKVSASHLE | 36 |
| tetani TT2 Th | | |
| Clostridium | KFIIKRYTPNNEIDSF | 37 |
| tetani TT3 Th | | |
| Clostridium | VSIDKFRIFCKALNPK | 38 |
| tetani TT4Th | | |
| Clostridium | WVRDIIDDFTNESSQKT | 39 |
| tetani2 Th | | |
| Diphtheria Th | DSETADNLEKTVAALSILPGHGC | 40 |
| EBV BHRF1 Th | AGLTLSLLVICSYLFISRG | 41 |
| EBV EBNA-1 Th | PGPLRESIVCYFMVFLQTHI | 42 |
| EBV CP Th | VPGLYSPCRAFFNKEELL | 43 |
| EBV GP340 Th | TGHGARTSTEPTTDY | 44 |
| EBV BPLF1 Th | KELKRQYEKKLRQ | 45 |
| EBV EBNA-2 | TVFYNIPPMPL | 46 |
| HCMV IE1 Th | DKREMWMACIKELH | 47 |
| Influenza | FVFTLTVPSER | 48 |
| MP1_1 Th | | |
| Influenza | SGPLKAEIAQRLEDV | 49 |
| MP1_2 Th | | |
| Influenza | DRLRRDQKS | 50 |
| NSP1 Th | | |
| Plasmodium | DHEKKHAKMEKASSVFNVVNS | 51 |
| falciparum Th | | |
| Schistosoma | KWFKTNAPNGVDEKHRH | 52 |
| mansoni Th | | |

TABLE 2
Examples of Optional Heterologous Spacers

| | | SEQ |
|---|---|---|
| Description | Sequence/Composition | ID NO |
| Naturally- | Naturally-occurring amino acids include: | N/A |
| Occurring | alanine, arginine, asparagine, aspartic acid, cysteine, glutamic | |
| Amino Acids | acid, glutamine, glycine, histidine, isoleucine, leucine, lysine, | |
| | methionine, phenylalanine, proline, serine, threonine, tryptophan, | |
| | tyrosine and valine | |
| Non-Naturally- | Non-naturally occurring amino acids include, but are not limited to: | N/A |
| Occurring | ε-N Lysine, β-alanine, ornithine, norleucine, norvaline, | |
| Amino Acids | hydroxyproline, thyroxine, γ-amino butyric acid, homoserine, | |
| | citrulline, aminobenzoic acid, 6-aminocaproic acid (Aca; 6- | |
| | Aminohexanoic acid), hydroxyproline, mercaptopropionic acid | |
| | (MPA), 3-nitro-tyrosine, pyroglutamic acid, and the like | |
| Chemicals | —NHCH(X)CH₂SCH₂CO—, | N/A |
| | —NHCH(X)CH₂SCH₂CO(εN)Lys-, | |
| | —NHCH(X)CH₂S-succinimidyl(εN)Lys-, | |
| | —NHCH(X)CH₂S-(succinimidyl)- | |
| Gly-Gly | -GG- | N/A |
| Epsilon-N | ε-K | N/A |
| Lysine | | |
| Epsilon-N | ε-K-KKK | 53 |
| Lysine-KKK | | |
| KKK-Epsilon-N | KKK-ε-K | 54 |
| Lysine | | |
| Hinge Sequence | Pro-Pro-Xaa-Pro-Xaa-Pro | 55 |

TABLE 3
Examples of Target Antigenic
Sites (B-Cell Epitopes)

| | | SEQ |
|---|---|---|
| | | ID |
| Description | Sequence | NO |
| Aβ₁₋₁₄ | DAEFRHDSGYEVHH | 56 |
| Aβ₁₋₁₆ | DAEFRHDSGYEVHH | 57 |
| | QK | |
| Aβ₁₇₋₄₂ | LVFFAEDVGSNKGA | 58 |
| | IIGLMVGGWIA | |
| Aβ₁₋₂₈ | DAEFRHDSGYEVHH | 59 |
| | QKLVFFAEDVGSNK | |
| Aβ₁₋₄₂ | DAEFRHDSGYEVHH | 60 |
| | QKLVFFAEDVGSNK | |
| | GAIIGLMVGGWIA | |
| α-Syn₁₂₆₋₁₃₅ | EMPSEEGYQD | 61 |
| (Derived | | |
| from | | |
| GenBank: | | |
| NP_000336) | | |
| IgE EMPD₁₋₃₉ | GLAGGSAQSQRAPD | 62 |
| | RVLCHSGQQQGLPR | |
| | AAGGSVPHPRC | |
| TaU₃₇₉₋₄₀₈ | RENAKAKTDHGAEIV | 63 |
| (Derived | YKSPWSGDTSPRHL | |
| from | | |
| GenBank: | | |
| AGF19246.1) | | |
| IL 3₁₉₇₋₁₄₄ | LSDKNIIDKIIEQLD | 64 |
| (Derived | KLKFQHEPETEISVP | |
| from | ADTFECKSFILTILQ | |
| Uniprot | QFS | |
| C7G0W1-1; | | |
| GenBank: | | |
| BAH97742.1) | | |

TABLE 4
Exemplary Peptide Immunogen Constructs

| De- | | SEQ |
|---|---|---|
| scrip- | | ID |
| tion | Sequence | NO |
| Aβ₁₋₁₄- | DAEFRHDSGYEVHH-ϵK-KKK- | 65 |
| ϵK- | ISISEIKGVIVHKIETILF | |
| KKK- | T RT TR | |
| MvF4 | | |
| Th | | |
| Aβ₁₋₁₄- | DAEFRHDSGYEVHH-ϵK-KKK- | 66 |
| ϵK- | ITITRIITIPQSLD | |
| HBsAg2 | FFLL L ITTI | |
| Th | | |
| Aβ₁₋₁₄- | DAEFRHDSGYEVHH-ϵK-KKK- | 67 |
| ϵK- | ISITEIKGVIVHRIETILF | |
| KKK- | | |
| MvF5 | | |
| Th | | |
| Aβ₁₋₁₄- | DAEFRHDSGYEVHH-ϵK-KKK-IITITRIITIITTID | 68 |
| ϵK- | | |
| HBsAg3 | | |
| Th | | |

TABLE 5
Exemplary CpG Oligonucleotides

| | | SEQ |
|---|---|---|
| | | ID |
| Description | Sequence/Composition | NO |
| CpG1 | 5′ TCg TCg TTT TgT CgT TTT | 69 |
| | gTC gTT TTg TCg TT 3′ | |
| | (fully | |
| | phosphorothioated) | |
| CpG2 | Phosphate TCg TCg TTT TgT | 70 |
| | CgT TTT gTC gTT 3′ | |
| | (fully | |
| | phosphorothioated) | |
| CpG3 | 5′ TCg TCg TTT TgT CgT TTT | 71 |
| | gTC gTT 3′ | |
| | (fully | |
| | phosphorothioated) | |

TABLE 6
Exclusive immunogenicity of Aβ₁₋₁₄ immunogens in guinea
pigs that target Aβ peptides but not Th epitopes

| | | Antibody titer at Week 8 (log₁₀) | | |
|---|---|---|---|---|
| | | Aβ₁₋₄₂ | MvF5 Th | HBsAg3 Th |
| | Animal | (SEQ ID | (SEQ ID | (SEQ ID |
| Peptide Immunogens | no. | NO: 60) | NO: 17) | NO: 28) |
| Aβ₁₋₁₄-εKKK-MvF5 Th | 1 | 4.68 | 0.21 | 0.31 |
| (SEQ ID NO: 67) + | 2 | 3.88 | 0.33 | 0.42 |
| Aβ₁₋₁₄-εK-HBsAg3 Th | 3 | 3.92 | 0.43 | 0.31 |
| (SEQ ID NO: 68) | 4 | 3.58 | 0.54 | 0.55 |
| | 5 | 3.35 | 0.52 | 0.38 |
| | 6 | 3.48 | 0.40 | 0.42 |
| | Mean | 3.82 | 0.41 | 0.39 |
| | (SD) | (0.48) | (0.12) | (0.09) |

TABLE 7
Measurement of cytokine concentration in Cynomolgus
Macaque Peripheral Blood Mononuclear Cells (PBMCs) upon stimulation
with Aβ₁₋₁₄, Aβ₁₋₄₂ peptides or PHA (Phytohemagglutin)
mitogen

| | | Cytokine Concentration^{a} (pg/mL) | | |
|---|---|---|---|---|
| | | Aβ₁₋₁₄ | Aβ₁₋₄₂ | |
| | | (SEQ ID | (SEQ ID | |
| Cytokine | Vaccine dose | NO: 56) | NO: 58) | PHA |
| IL-2 | Placebo | BDL^{b} | 23.3 ± 13.1 | 90.6 ± 12.4 |
| | 150 μg | BDL | 19.4 ± 9.7 | 96.1 ± 13.3 |
| | 750 μg | BDL | 25.2 ± 11.8 | 97.5 ± 6.6 |
| IL-6 | Placebo | BDL | 23.1 ± 11.7 | 69.1 ± 12.0 |
| | 150 μg | BDL | 15.0 ± 9.1 | 70.6 ± 15.7 |
| | 750 μg | BDL | 23.4 ± 10.5 | 66.2 ± 7.3 |
| TNF-α | Placebo | BDL | 9.2 ± 5.3 | 91.0 ± 29.1 |
| | 150 μg | BDL | 7.9 ± 4.8 | 96.1 ± 22.2 |
| | 750 μg | BDL | 7.8 ± 5.9 | 89.0 ± 13.7 |

^{a}Result was shown as mean ± standard deviation
^{b}BDL, below detection level

TABLE 8
Stimulation Index of peripheral blood mononuclear cells
evaluated from the 19 patients with Alzheimer's disease

| | Week 0 | | Week 16 | | Difference | | Paired t-test |
|---|---|---|---|---|---|---|---|
| Peptide | Mean (SD) | | Mean (SD) | | Mean (SD) | | p value |
| Aβ₁₋₁₄ | 0.93 | (0.36) | 0.90 | (0.22) | −0.03 | (0.39) | 0.73 |
| (SEQ ID NO: 56) | | | | | | | |
| Aβ₁₋₁₆ | 0.92 | (0.30) | 0.98 | (0.25) | 0.06 | (0.40) | 0.54 |
| (SEQ ID NO: 57) | | | | | | | |
| Aβ₁₋₂₈ | 0.96 | (0.30) | 1.04 | (0.34) | 0.08 | (0.56) | 0.55 |
| (SEQ ID NO: 59) | | | | | | | |
| Aβ₁₇₋₄₂ | 0.96 | (0.34) | 1.04 | (0.29) | 0.08 | (0.49) | 0.47 |
| (SEQ ID NO: 58) | | | | | | | |
| Aβ₁₋₄₂ | 0.97 | (0.38) | 1.08 | (0.49) | 0.10 | (0.53) | 0.40 |
| (SEQ ID NO: 60) | | | | | | | |
| p1412 | 0.87 | (0.22) | 0.99 | (0.33) | 0.11 | (0.34) | 0.18 |
| (non-relevant peptide) | | | | | | | |
| PHA | 28.73 | (14.2) | 27.75 | (32.9) | −0.98 | (26.6) | 0.87 |

TABLE 9
Cytokine concentrations in human peripheral blood mononuclear
cells (PBMC) upon stimulation with Aβ peptides or PHA mitogen¹

| | Th1 | | | | | | | | Th 1 | | | |
|---|---|---|---|---|---|---|---|---|---|---|---|---|
| | IL2 | | | | IFN-γ | | | | IL-6 | | | |
| Peptide | W 0 | | W 16 | | W 0 | | W 16 | | W 0 | | W 16 | |
| Aβ₁₋₁₄ | 31.1 | (32.5) | 31.2 | (24.3) | 13.5 | (16.9) | 16.1 | (12.9) | 31.3 | (29.7) | 50.7 | (52.0) |
| (SEQ ID | | | | | | | | | | | | |
| NO: 56) | | | | | | | | | | | | |
| Aβ₁₋₁₆ | 31.4 | (31.4) | 36.0 | (23.9) | 15.0 | (16.1) | 13.8 | (14.2) | 52.5 | (31.7) | 50.4 | (42.6) |
| (SEQ ID | | | | | | | | | | | | |
| NO: 57) | | | | | | | | | | | | |
| Aβ₁₋₂₈ | 36.7 | (34.3) | 40.6 | (28.0) | 16.0 | (23.6) | 20.7 | (24.4) | 31.7 | (25.4) | 42.3 | (41.8) |
| (SEQ ID | | | | | | | | | | | | |
| NO: 59) | | | | | | | | | | | | |
| Aβ₁₇₋₄₂ | 24.6 | (25.7) | 29.2 | (21.2) | 9.7 | (9.7) | 13.6 | (15.6) | >44.6 | (70.9)³ | 46.9 | (51.3) |
| (SEQ ID | | | | | | | | | | | | |
| NO: 58) | | | | | | | | | | | | |
| Aβ₁₋₄₂ | 23.1 | (17.7) | 27.3 | (16.9) | 13.4 | (16.1) | >44.8 | (77.3) | >141 | (130)⁴ | >202 | (121)⁵ |
| (SEQ ID | | | | | | | | | | | | |
| NO: 60) | | | | | | | | | | | | |
| p1412 | 30.9 | (27.4) | 40.0 | (26.0) | 14.4 | (18.4) | 21.7 | (30.0) | 31.8 | (52.1) | 60.7 | (95.8) |
| PHA | 10.4⁷ | (11.3) | 12.8⁷ | (6.5) | >320 | (0.00)² | >319 | (4.8)² | >320 | (0.00)² | >320 | (0.00)² |
| Cell | 33.4 | (24.9) | 38.8 | (33.1) | 13.8 | (12.3) | 17.8 | (18.2) | 45.9 | (41.9) | 65.3 | (76.5) |
| control | | | | | | | | | | | | |

| | Th 1 | | | | Both | | | |
|---|---|---|---|---|---|---|---|---|
| | IL-10 | | | | TNF-α | | | |
| Peptide | W 0 | | W 16 | | W 0 | | W 16 | |
| Aβ₁₋₁₄ | 5.7 | (1.6) | 5.6 | (1.6) | 36.8 | (62.8) | 39.8 | (51.7) |
| (SEQ ID | | | | | | | | |
| NO: 56) | | | | | | | | |
| Aβ₁₋₁₆ | 5.7 | (1.6) | 5.8 | (1.8) | 47.4 | (72.2) | 47.23 | (69.7) |
| (SEQ ID | | | | | | | | |
| NO: 57) | | | | | | | | |
| Aβ₁₋₂₈ | 5.6 | (1.5) | 6.2 | (2.5) | 41.6 | (66.5) | 51.2 | (67.8) |
| (SEQ ID | | | | | | | | |
| NO: 59) | | | | | | | | |
| Aβ₁₇₋₄₂ | 5.3 | (0.86) | 5.6 | (1.5) | 15.6 | (18.4) | 24.8 | (39.3) |
| (SEQ ID | | | | | | | | |
| NO: 58) | | | | | | | | |
| Aβ₁₋₄₂ | 11.1 | (22.7) | 31.9 | (50.2) | >31.6 | (71.5)³ | >88.8 | (133)⁶ |
| (SEQ ID | | | | | | | | |
| NO: 60) | | | | | | | | |
| p1412 | 5.3 | (0.64) | 5.2 | (0.53) | 17.1 | (23.5) | 20.9 | (29.3) |
| PHA | 174 | (84.8) | >163 | (99.7) | >313 | (30.5)² | >301 | (46.5)² |
| Cell | 5.9 | (2.5) | 5.7 | (1.6) | 44.3 | (70.9) | 46.7 | (67.8) |
| control | | | | | | | | |

¹Quantifiable range of the assay is between 5 and 320 pg/mL
²Concentration of >90% subjects were above the upper quantification limit (AQL > 320 pg/mL)
³One patient had an AQL value
⁴Six patients had AQL values
⁵Eight patients had AQL values
⁶Four patients had AQL values
⁷The lack of IL-2 production observed in response to PHA mitogen was consistent with data reported under similar experimental conditions

1. A method of treating a condition comprising administering a peptide to a subject in need thereof, wherein the peptide comprises a T helper cell epitope and an antigen-presenting epitope, wherein the peptide produces an immunogenic inflammatory response that is at least about 3-fold lower than an immunogenic inflammatory response of a positive control.

2.-63. (canceled)

64. The method of claim 1, wherein
(a) the antigen-presenting epitope is a B cell epitope or a peptide hapten and
(b) the T helper cell epitope is a Th1 epitope or Th2 epitope.

65. The method of claim 1, wherein the T helper cell epitope is an artificial T cell epitope.

66. The method of claim 1, wherein the T helper cell epitope is selected from the group consisting of SEQ ID NOs: 1-52.

67. The method of claim 1, wherein the T helper cell epitope and the antigen-presenting epitope are covalently linked.

68. The method of claim 1, wherein the T helper cell epitope is attached to the antigen-presenting epitope through a spacer.

69. The method of claim 68, wherein the spacer is Gly-Gly, (ε-N)Lys, or any one of SEQ ID NOs: 53-55.

70. The method of claim 1, wherein the immunologic inflammatory response is measured in peripheral blood mononuclear cells or isolated peripheral blood mononuclear cells.

71. The method of claim 1, wherein the immunologic inflammatory response is an increase in cytokine concentration.

72. The method of claim 71, wherein the increase in cytokine concentration is an increase in concentration of IL-2, IL-6, IL-10, INF-γ, or TNF-α.

73. The method of claim 1, wherein the positive control is a phytohaemagglutinin mitogen.

74. The method of claim 1, wherein administering comprises administering about 150 μg to about 750 μg of the peptide.

75. The method of claim 1, wherein the peptide is of the formula:
(A)ₙ-(Target antigenic site)-(B)ₒ-(Th)ₘ-X
or
(A)ₙ-(B)ₒ-(Th)ₘ-(B)ₒ-(Target antigenic site)-X
or
(A)ₙ-(Th)ₘ-(B)ₒ-(Target antigenic site)-X
or
(Target antigenic site)-(B)ₒ-(Th)ₘ-(A)ₙ-X
or
(Th)ₘ-(B)ₒ-(Target antigenic site)-(A)ₙ-X
wherein:
A is an amino acid or an immunostimulatory sequence;
B is at least one amino acid, —NHCH(X)CH₂SCH₂CO—, —NHCH(X)CH₂SCH₂CO(εN)Lys-, —NHCH(X)CH₂S-succinimidyl(εN)Lys-, or —NHCH(X)CH₂S-(succinimidyl)-;
Th is a helper T cell epitope, an analog, or a segment thereof;
Target antigenic site is a B cell epitope or an immunologically reactive analogue thereof;
X is an amino acid α-COOH, —CONH₂;
n is from 1 to about 10;
m is from 1 to about 4; and
o is from 0 to about 10.

76. The method of claim 1, wherein the peptide produces an immunogenic inflammatory response in the subject that is less than about 3-fold higher than an immunogenic inflammatory response of a negative control.


SEQUENCE LISTING (71 sequences)

SEQ ID NO: 1 - protein, 16 aa, Measles virus
  PEPTIDE (1)..(16): MvF Th
DLSDLKGLLLHKLDGL

SEQ ID NO: 2 - protein, 16 aa, Measles virus
  PEPTIDE (1)..(16): MvF Th
EISDEIRLIIIKRIEI

SEQ ID NO: 3 - protein, 16 aa, Measles virus
  PEPTIDE (1)..(16): MvF Th
DVSDVKGVVVHKVDGV

SEQ ID NO: 4 - protein, 16 aa, Measles virus
  PEPTIDE (1)..(16): MvF Th
DFSDFKGFFFHKFDGF

SEQ ID NO: 5 - protein, 16 aa, Measles virus
  PEPTIDE (1)..(16): MvF Th
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 6 - protein, 15 aa, Measles virus
  PEPTIDE (1)..(15): MvF1 Th
LSEIKGVIVHRLEGV

SEQ ID NO: 7 - protein, 15 aa, Measles virus
  PEPTIDE (1)..(15): MvF 2 Th
ISEIKGVIVHKIEGI

SEQ ID NO: 8 - protein, 19 aa, Measles virus
  PEPTIDE (1)..(19): MvF3 Th
ISISEIKGVIVHKIEGILF

SEQ ID NO: 9 - protein, 19 aa, Measles virus
  PEPTIDE (1)..(19): MvF 3Th
ISITEIRTVIVTRIETILF

SEQ ID NO: 10 - protein, 19 aa, Measles virus
  PEPTIDE (1)..(19): MvF3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIETILF

SEQ ID NO: 11 - protein, 22 aa, Measles virus
  PEPTIDE (1)..(22): KKKMvF3 Th
KKKISISEIKGVIVHKIEGILF

SEQ ID NO: 12 - protein, 22 aa, Measles virus
  PEPTIDE (1)..(22): KKKMvF3 Th
KKKISITEIRTVIVTRIETILF

SEQ ID NO: 13 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 14 - protein, 19 aa, Measles virus
  PEPTIDE (1)..(19): MvF4 Th
ISISEIKGVIVHKIETILF

SEQ ID NO: 15 - protein, 19 aa, Measles virus
  PEPTIDE (1)..(19): MvF4 Th
ISITEIRTVIVTRIETILF

SEQ ID NO: 16 - protein, 19 aa, Measles virus
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 17 - protein, 19 aa, Measles virus
  PEPTIDE (1)..(19): MvF5 Th (UBITh1)
ISITEIKGVIVHRIETILF

SEQ ID NO: 18 - protein, 22 aa, Measles virus
  PEPTIDE (1)..(22): KKKMvF5 Th (UBITh1a)
KKKISITEIKGVIVHRIETILF

SEQ ID NO: 19 - protein, 18 aa, Hepatitis B virus
  PEPTIDE (1)..(18): HBsAg1 Th
KKKLFLLTKLLTLPQSLD

SEQ ID NO: 20 - protein, 18 aa, Hepatitis B virus
  PEPTIDE (1)..(18): HBsAg1 Th
RRRIKIITRIITIPLSIR

SEQ ID NO: 21 - protein, 18 aa, Hepatitis B virus
  PEPTIDE (1)..(18): HBsAg1 Th
KKKVRVVTKVVTVPISVD

SEQ ID NO: 22 - protein, 18 aa, Hepatitis B virus
  PEPTIDE (1)..(18): HBsAg1 Th
KKKFFFFTKFFTFPVSFD

SEQ ID NO: 23 - protein, 18 aa, Hepatitis B virus
  PEPTIDE (1)..(18): HBsAg1 Th
KKKLFLLTKLLTLPFSLD

SEQ ID NO: 24 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 25 - protein, 18 aa, Hepatitis B virus
  PEPTIDE (1)..(18): HBsAg2 Th
KKKIITITRIITIPQSLD

SEQ ID NO: 26 - protein, 18 aa, Hepatitis B virus
  PEPTIDE (1)..(18): HBsAg2 Th
KKKFFLLTRILTIITTID

SEQ ID NO: 27 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 28 - protein, 18 aa, Hepatitis B virus
  PEPTIDE (1)..(18): HBsAg 3 Th
KKKIITITRIITIITTID

SEQ ID NO: 29 - protein, 15 aa, Hepatitis B virus
  PEPTIDE (1)..(15): HBsAg4 Th (UBITh4)
FFLLTRILTIPQSLD

SEQ ID NO: 30 - protein, 18 aa, Hepatitis B virus
  PEPTIDE (1)..(18): KKK-HBsAg Th
KKKFFLLTRILTIPQSLD

SEQ ID NO: 31 - protein, 14 aa, Hepatitis B virus
  PEPTIDE (1)..(14): HBsAg Th
FFLLTRILTIPQSL

SEQ ID NO: 32 - protein, 24 aa, Bordetella pertussis
  PEPTIDE (1)..(24): Bordetella pertussis Th
GAYARCPNGTRALTVAELRGNAEL

SEQ ID NO: 33 - protein, 25 aa, Cholera Toxin
  PEPTIDE (1)..(25): Cholera Toxin Th
ALNIWDRFDVFCTLGATTGYLKGNS

SEQ ID NO: 34 - protein, 15 aa, Clostridium tetani
  PEPTIDE (1)..(15): Clostridium tetani TT1 Th
QYIKANSKFIGITEL

SEQ ID NO: 35 - protein, 17 aa, Clostridium tetani
  PEPTIDE (1)..(17): Clostridium tetani 1 Th
KKQYIKANSKFIGITEL

SEQ ID NO: 36 - protein, 21 aa, Clostridium tetani
  PEPTIDE (1)..(21): Clostridium tetani  TT2 Th
FNNFTVSFWLRVPKVSASHLE

SEQ ID NO: 37 - protein, 16 aa, Clostridium tetani
  PEPTIDE (1)..(16): Clostridium tetani TT3 Th
KFIIKRYTPNNEIDSF

SEQ ID NO: 38 - protein, 16 aa, Clostridium tetani
  PEPTIDE (1)..(16): Clostridium tetani  TT4 Th
VSIDKFRIFCKALNPK

SEQ ID NO: 39 - protein, 17 aa, Clostridium tetani
  PEPTIDE (1)..(17): Clostridium tetani 2 Th
WVRDIIDDFTNESSQKT

SEQ ID NO: 40 - protein, 23 aa, diphtheria bacilli
  PEPTIDE (1)..(23): Diphtheria Th
DSETADNLEKTVAALSILPGHGC

SEQ ID NO: 41 - protein, 19 aa, Epstein-Barr virus
  PEPTIDE (1)..(19): EBV BHRF1 Th
AGLTLSLLVICSYLFISRG

SEQ ID NO: 42 - protein, 20 aa, Epstein-Barr virus
  PEPTIDE (1)..(20): EBV EBNA-1 Th
PGPLRESIVCYFMVFLQTHI

SEQ ID NO: 43 - protein, 18 aa, Epstein-Barr virus
  PEPTIDE (1)..(18): EBV CP Th
VPGLYSPCRAFFNKEELL

SEQ ID NO: 44 - protein, 15 aa, Epstein-Barr virus
  PEPTIDE (1)..(15): EBV GP340 Th
TGHGARTSTEPTTDY

SEQ ID NO: 45 - protein, 13 aa, Epstein-Barr virus
  PEPTIDE (1)..(13): EBV BPLF1 Th
KELKRQYEKKLRQ

SEQ ID NO: 46 - protein, 11 aa, Epstein-Barr virus
  PEPTIDE (1)..(11): EBV EBNA-2 Th
TVFYNIPPMPL

SEQ ID NO: 47 - protein, 14 aa, Human cytomegalovirus
  PEPTIDE (1)..(14): HCMV IE1 Th
DKREMWMACIKELH

SEQ ID NO: 48 - protein, 11 aa, Influenza virus
  PEPTIDE (1)..(11): Influenza Matrix protein 1 _1 Th
  PEPTIDE (1)..(11): Influenza Matrix protein 1_1 Th
FVFTLTVPSER

SEQ ID NO: 49 - protein, 15 aa, Influenza virus
  PEPTIDE (1)..(15): Influenza Matrix protein 1_2 Th
SGPLKAEIAQRLEDV

SEQ ID NO: 50 - protein, 9 aa, Influenza virus
  PEPTIDE (1)..(9): Influenza Non-structural protein 1 Th
DRLRRDQKS

SEQ ID NO: 51 - protein, 21 aa, Plasmodium falciparum
  PEPTIDE (1)..(21): Plasmodium falciparum Th
DHEKKHAKMEKASSVFNVVNS

SEQ ID NO: 52 - protein, 17 aa, Schistosoma mansoni
  PEPTIDE (1)..(17): Schistosoma mansoni Th
KWFKTNAPNGVDEKHRH

SEQ ID NO: 53 - protein, 4 aa, Artificial Sequence
  Synthetic peptide
  SITE (1)..(1): epsilon-K
  PEPTIDE (1)..(4): epsilon-K-KKK as a spacer
KKKK

SEQ ID NO: 54 - protein, 4 aa, Artificial Sequence
  Synthetic peptide
  PEPTIDE (1)..(4): KKK-epsilon-K as a spacer
  SITE (4)..(4): epsilon-K
KKKK

SEQ ID NO: 55 - protein, 6 aa, Artificial Sequence
  Synthetic peptide
  PEPTIDE (1)..(6): Flexible Hinge Spacer
PPXPXP

SEQ ID NO: 56 - protein, 14 aa, Homo sapiens
  PEPTIDE (1)..(14): Abeta 1-14
DAEFRHDSGYEVHH

SEQ ID NO: 57 - protein, 16 aa, Homo sapiens
  PEPTIDE (1)..(16): Abeta 1-16
DAEFRHDSGYEVHHQK

SEQ ID NO: 58 - protein, 26 aa, Homo sapiens
  PEPTIDE (1)..(26): Abeta 17-42
LVFFAEDVGSNKGAIIGLMVGGVVIA

SEQ ID NO: 59 - protein, 28 aa, Homo sapiens
  PEPTIDE (1)..(28): Abeta 1-28
DAEFRHDSGYEVHHQKLVFFAEDVGSNK

SEQ ID NO: 60 - protein, 42 aa, Homo sapiens [sequence omitted]

SEQ ID NO: 61 - protein, 10 aa, Homo sapiens
  PEPTIDE (1)..(10): Alpha-Syn 126-135 (Derived from GenBank NP_000336)
EMPSEEGYQD

SEQ ID NO: 62 - protein, 39 aa, Homo sapiens
  PEPTIDE (1)..(39): IgE-EMPD1-39
GLAGGSAQSQRAPDRVLCHSGQQQGLPRAAGGSVPHPRC

SEQ ID NO: 63 - protein, 30 aa, Homo sapiens
  PEPTIDE (1)..(30): Tau 379-408 (Derived from GenBank AGF19246.1)
RENAKAKTDHGAEIVYKSPVVSGDTSPRHL

SEQ ID NO: 64 - protein, 48 aa, Homo sapiens
  PEPTIDE (1)..(48): IL-31 97-144 (Derived from Uniprot C7G0W1-1; GenBank BAH97742.1)
LSDKNIIDKIIEQLDKLKFQHEPETEISVPADTFECKSFILTILQQFS

SEQ ID NO: 65 - protein, 37 aa, Artificial Sequence
  Synthetic peptide
  PEPTIDE (1)..(14): Abeta 1-14
  PEPTIDE (15)..(18): epsilon-K-KKK as a spacer
  SITE (15)..(15): epsilon-K
  PEPTIDE (19)..(37): MvF 4 Th
  SITE (22)..(22): S or T
  SITE (25)..(25): K or R
  SITE (26)..(26): G or T
  SITE (30)..(30): H or T
  SITE (31)..(31): K or R
DAEFRHDSGYEVHHKKKKISIXEIXXVIVXXIETILF

SEQ ID NO: 66 - protein, 33 aa, Artificial Sequence
  Synthetic peptide
  PEPTIDE (1)..(14): Abeta 1-14
  SITE (15)..(15): epsilon-K
  PEPTIDE (16)..(33): HBsAg 2 Th
  SITE (19)..(19): I or F
  SITE (20)..(20): I or F
  SITE (21)..(21): T or L
  SITE (22)..(22): I or L
  SITE (26)..(26): I or L
  SITE (29)..(29): P or I
  SITE (30)..(30): Q or T
  SITE (31)..(31): S or T
  SITE (32)..(32): L or I
DAEFRHDSGYEVHHKKKKXXXXTRIXTIXXXXD

SEQ ID NO: 67 - protein, 37 aa, Artificial Sequence
  Synthetic peptide
  PEPTIDE (1)..(14): Abeta 1-14
  SITE (15)..(15): epsilon K
  PEPTIDE (15)..(18): epsilon K-KKK as a spacer
  PEPTIDE (19)..(37): MvF5 Th (UBITh1)
DAEFRHDSGYEVHHKKKKISITEIKGVIVHRIETILF

SEQ ID NO: 68 - protein, 33 aa, Artificial Sequence
  Synthetic peptide
  PEPTIDE (1)..(14): Abeta 1-14
  SITE (15)..(15): epsilon K
  PEPTIDE (16)..(33): HBsAg3 Th
DAEFRHDSGYEVHHKKKKIITITRIITIITTID

SEQ ID NO: 69 - DNA, 32 bp, Artificial Sequence
  CpG1 oligonucleotide ODN
tcgtcgttttgtcgttttgtcgttttgtcgtt

SEQ ID NO: 70 - DNA, 24 bp, Artificial Sequence
  CpG2 oligonucleotide ODN
  misc_feature (1)..(1): phosphorothioate group
tcgtcgttttgtcgttttgtcgtt

SEQ ID NO: 71 - DNA, 24 bp, Artificial Sequence
  CpG3 Oligonucleotide ODN
tcgtcgttttgtcgttttgtcgtt